(19) United States
(12) Patent Application Publication
(10) Pub. No.: US 2007/0207179 A1
(43) Pub. Date: Sep. 6, 2007

(54) Medical Device

(76) Inventors: Erik Andersen, Roskilde (DK); Daniel Smith, Stow, OH (US); Darrell Reneker, Akron, OH (US)

Correspondence Address: ROETZEL AND ANDRESS, 222 SOUTH MAIN STREET, AKRON, OH, 44308, US

(21) Appl. No.: 10/595,339

(22) PCT Filed: Oct. 14, 2004
(86) PCT No.: PCT/US04/33949

§ 371 Date: Dec. 29, 2006

Related U.S. Application Data
(60) Provisional application No. 60/510,520, filed on Oct. 14, 2003.
(60) Provisional application No. 60/529,629, filed on Dec. 16, 2003.
(60) Provisional application No. 60/566,087, filed on Apr. 29, 2004.

(30) Foreign Application Priority Data
Oct. 14, 2003 (DK) PA 2003 01514
Dec. 16, 2003 (DK) PA 2003 01864
Apr. 29, 2004 (DK) PA 2004 00671

(51) Int. Cl. A61F 2/06; A61K 31/66; A61L 27/00; A61M 5/00

(52) U.S. Cl. 424/423; 424/426; 514/149; 523/105; 604/265; 606/200; 623/1.1; 623/1.43; 623/1.49

(57) ABSTRACT

A medical device, such as a guide wire, an embolization device, or a guide shaft for a micro catheter, comprises a solid and/or non-expandable core member made from e.g. metal, such as tantalum, and an outer surface layer, which is formed by electrospun nanofibers. The outer surface layer may incorporate a pharmaceutically active substance, such as a nitric oxide (NO) donor for release in the vascular or neurovascular system of a living being. The NO donor may be incorporated in a polymer, such as a polymeric linear poly(ethylenimine) diazeniumdiolate.

### TECHNICAL FIELD

The present invention relates to a medical device and its method of manufacture, in particular a guide wire or an embolization device.

### BACKGROUND OF THE INVENTION

Medical devices, such as guide wires and embolization devices are often used in various diagnostic procedures and medical treatments. The devices often contain drugs that after implantation elute to the surrounding tissue as to avoid side effects such as cell proliferation. It is generally desired that medical devices for insertion into the vascular system of a living being meet certain physical requirements. For example, the medical devices must be able to conform to an often tortuous passage to the treatment site while being sufficiently rigid to enable secure insertion. Furthermore, the surfaces of such medical devices should be hydrophilic and have a low surface friction in order to facilitate introduction. The surfaces may be coated with nitric oxide containing polymer matrix. Such Nitric oxide releasing matrixes may relax or prevent arterial spasm once the medical device is in place. Medical devices which are intended to release drugs once inserted into the vascular system of a living being may be covered or coated with appropriate pharmaceutical compounds. Expandable stents are often placed on an angloplasty balloon catheter which, once in place, is inflated in order to cause the stent to expand. Alternatively, stents may be made from a material which has a recovery capacity such as a super elastic alloy, such as Nitinol, so that the stents may automatically expand, once in place. Such self expanding stents are often delivered by a telescopic tube arrangement where an outer member is removed e.g. by forced sliding over an inner member to which the stent is fixed prior to expansion.

Embolization devices are e.g. employed in order to block blood supply to regions of tumors or in the treatment of anorysms.

In the prior art, various medical devices, including stents and catheters, as well as methods for their manufacture have been proposed. U.S. Pat. No. 6,030,371 discloses a method for nonextrusion manufacturing of catheters that can be used to produce catheters. A polymer material in a particulate preform is applied in a layer over an outer surface of a core member. By applying the layer in a particulate preform, a composition of the polymer material can be varied continuously as it is being applied to provide a variable hardness over the length of the catheter. A fibrous reinforcement can be used having a constant or variable pitch and a constant or variable number of fibers and fiber types may be employed. U.S. Pat. No. 6,030,371 further discloses the use of a plurality of mandrels placed side-by-side to form a multiple lumen tubing.

Various nictric oxide (NO) donor compounds, pharmaceutical compositions containing such nitric oxide donor compounds and polymeric compositions capable of releasing nitric oxide have also been proposed in the prior art. For example, European patent No. 1220694 B1 corresponding to U.S. Pat. No. 6,737,447 B1 discloses a medical device comprising at least one nanofiber of a linear poly(ethylenimine) diazeniumdiolate forming a coating layer on the device. This polymer is effective in delivering nitric oxide to tissues surrounding medical device. EP 1220694 B1 mentions the possibility of depositing the polymer by an electrospinning process.

### SUMMARY OF THE INVENTION

It is an object of preferred embodiments of the present invention to provide a coated solid and/or non-expandable medical device, such as a guide wire or an embolization device, the manufacture of which may be accurately controlled. It is a further object of preferred embodiments to provide such a device which is coated with a material capable of conveying an efficient amount of a pharmaceutically active substance to a treatment site and efficiently releasing the substance at the treatment site.

In a first aspect, the invention provides a medical device comprising a solid and/or non-expandable core member having an outer surface layer, which is formed by electrospun nanofibers. The invention also provides a method of producing a medical device comprising a solid and/or non-expandable core member having an outer surface-layer, the method comprising forming the outer surface layer by electrospinning of nanofibers.

The present inventors have realized that solid and/or non-expandable core members, such as wires or particles, in particular metal or polymer wires or particles, such as guide wires or embolization devices, may advantageously be coated with electrospun nanofibers, as such fibers offer a large surface area even on a small-diameter core member. Thus, such coatings are useful as reservoirs for drugs to be released at a treatment site, e.g. in the vascular or neurovascular system of a living being. Moreover, electrospinning offers great accuracy and results in devices with a low surface friction.

The core member may consist essentially of a string or a helical coil element which is preferably made from metal or from a polymer, such as a biodegradable polymer, such as from polylactidacid. A helical coil element may define any curved trajectory in space. For example, it may form a helical spring form or a so-called three-dimensional sphere in which the coil element extends in an apparently random fashion to match a cavity at the application site in the body of the living being. In certain embodiments, a further coil or three-dimensional sphere may be wound around a first coil which has the form of a helical spring. Coil elements are often employed as embolization devices.

Alternatively, the core member may comprise one or more particles, preferably metal particles, such as tantalum or tungsten particles, onto which filaments are applied by electrospinning. The particles may be provided on a film of a plastics material by which they are supported while being coated by the electrospinning process, or they may be coated with electrospun nanofibers in a fluid bed arrangement. The fluid bed may be arranged with an air stream at a negative potential with the source of electrospinning at a positive potential. Such particles provided with an electrospun filament may be injected into the body of a living being through a micro catheter, which also may be produced by electrospinning of nanofibers. Particles, to which there is applied an electronspun nano filament, are often employed as embolization devices.

It has been found that a fibrous surface or a thrombogenic material provided, e.g. on a coil member covered with nanospun fibers, may enhance formation of thrombus or embolization which is advantageous for curing arterial malfunctions in the vascular system.

Typically, the diameter of the nanofibers is in the range of 2 to 4000 nanometers, preferably 2 to 3000 nanometers, and accordingly a large number of nanofibers is present on the outer surface of the device. It will thus be appreciated that the nanofibers on the outer surface of the device define a large accumulated area, the area being larger with respect to the weight of the device than what is achievable with most other non-electrospun surfaces. Accordingly, the electrospun surface constitutes a relatively large reservoir for the pharmaceutically active substance compared to the weight of the coated device. Nanofibers may even be manufactured to a diameter of 0.5 nanometer which is close to the size of a single molecule.

It has been found that such spinning of nanofibers may be more easily or accurately controlled than methods relying solely on spraying of polymers toward a core. This may confer the further advantage that medical devices may be made with smaller dimensions, such as smaller diameters than hitherto. The present invention allows for the manufacture of devices with relatively low diameters which, in comparison to devices with larger diameters, facilitate introduction into the vascular system of a living being and reduce side-effects which may occur as a consequence of the introduction of the device. The spinning of nanofibers allows for the manufacture of integrated composite devices, in which two or more materials are interlocked on a molecular scale, in small dimensions while maintaining a sufficient mechanical stability. Cross-sectional dimensions as small as the dimension of approximately 2-5 molecules of the spun material may be achieved. The size of the molecules evidently depends from the source material used, the size of a polyurethane molecule being usually in the range of less than 3000 nanometers. It will thus be appreciated that devices may be manufactured with a much smaller diameter than hitherto, typical prior art stents having a diameter of order of magnitude 2 mm and larger.

It has also been found that devices produced by preferred embodiments of the method according to the invention have a low surface friction. In embodiments of the invention, a low surface friction may be achieved by applying a hygroscopic material as a fiber forming material for the electrospinning process. Accordingly, once introduced into the vascular system, the hygroscopic electrospun material absorbs bodily fluid, resulting in a hydrophilic low-friction surface. A hygroscopic surface may for example be achieved with a polyurethane or a polyacrylic acid material.

It should be understood that the term electrospinning comprises a process wherein particles are applied onto a base element which is kept at a certain, preferably constant, electric potential, preferably a negative potential. The particles emerge from a source which is at another, preferably positive potential. The positive and negative potentials may e.g. be balanced with respect to the potential of a surrounding environment, i.e. a room in which the process is being performed. The potential of the base element with respect to the potential of the surrounding atmosphere may preferably be between −5 and −30 kV, and the positive potential of the source with respect to the potential of the surrounding atmosphere may preferably be between +5 and +30 kV, so that the potential difference between source and base element is between 10 and 60 kV.

The art of electrospinning of nanofibers has developed considerably in recent years. U.S. Pat. No. 6,382,526 discloses a process and apparatus for the production of nanofibers, which process and apparatus are useful in the method according to the present invention, and U.S. Pat. No. 6,520,425 discloses a nozzle for forming nanofibers. It should be understood that the processes and apparatuses of the aforementioned US patents may be applicable in the method according to the present invention, but that the scope of protection is not restricted to those processes and apparatuses.

In case of an elongated device, e.g. a guide wire, it may define a plurality of sections along its length. For example, the sections may have different properties, such as different hardness. Such different properties may be arrived at by employing different fiber-forming materials for different sections and/or by changing production parameters, such as voltage of electrodes in the electrospinning process, distance between high-voltage and low-voltage electrodes, rotational speed of the device (or of a core wire around which the device is manufactured), electrical field intensity, corona discharge initiation voltage or corona discharge current.

The outer surface layer of the device may constitute a reservoirs to drugs. The electrospun portions thereof constitute reservoirs for holding drugs or constitute a matrix polymer source where the drug is either blocked into the molecule chain or adheres to or surrounds the molecule chain. The devices disclosed herein may carry any appropriate drug, including but not limited to nitric oxide compositions, heparin and chemotherapeutical agents.

A guide wire coated with an electrospun material incorporating e.g. nitric oxide may be useful for relaxing arterial walls when the guide wire is used for placing another medical device in the vascular system of a living being, e.g. a balloon and/or stent, or a stent graft.

Embolization devices may e.g. be formed from or incorporate a thrombogenic material, e.g. a biodegradable thrombogenic polymer. A biocompatible polyurethane and/or a polylactid may be used.

The outer surface layer of the device is preferably made from electrospun fibres which incorporate at least one pharmaceutically active substance. The electrospun fibres form a polymer matrix of one or more polymers. It should be understood that the “outer surface layer made from electrospun fibres, i.e. the polymer matrix, needs not to be the outermost layer of the device, for example a layer of a hydrophilic polymer (e.g. polyacrylic acids (and copolymers), polyethylene oxides, poly(N-vinyl lactams such as polyvinyl pyrrolidone, etc.) may be provided as a coating on the outer surface layer (polymer matrix). Alternatively, a barrier layer may be provided as coating on the outer surface layer (polymer matrix) in order to ensure that contact between the polymer matrix and blood is delayed until the device is in place. The barrier layer may either be formed of a biodegradable polymer which dissolves or disintegrates.

By the term “polymer matrix” is meant the three-dimensional structure formed by the electrospun fibers. Due to the nature of the electrospinning process, the polymer matrix is characterized by a very high accessible surface area which allows swift liberation of the pharmaceutically active substance(s). The polymer of the polymer matrix may be prepared from various polymer-based materials and composite matrixes thereof, including polymer solutions and polymer melts. Applicable polymers are, e.g., polyamides including nylon, polyurethanes, fluoropolymers, polyolefins, polyimides, polyimines, (meth)acrylic polymers, and polyesters, as well as suitable co-polymers. Further, carbon may be used as a fiber-forming material.

The polymer matrix is formed of one or more polymers and may—in addition to the pharmaceutically active substance(s)—incorporate or comprise other ingredients such as salts, buffer components, microparticles, etc.

By the term “incorporates at least one pharmaceutically active substance” is meant that the pharmaceutically active substance(s) is/are either present as discrete molecules within the polymer matrix or is/are bound to the polymer(s) of the matrix either by covalent bonds or by ionic interactions. In the latter of the two instances, the pharmaceutically active substance(s) typically needs to be liberated from the polymer molecules before the biological effect can enter into effect. Liberation will often take place upon contact with physiological fluids (e.g. blood) by hydrolysis, ion-exchange, etc.

In one preferred embodiment, the pharmaceutically active substance is covalently bound to polymer molecules.

The pharmaceutically active substance may be mixed into a liquid substance from which the outer surface layer is manufactured.

In one interesting embodiment, the pharmaceutically active substance is a nitric oxide donor. For certain medical treatments, it is desired that nitric oxide is released into the body tissue in the gas phase immediately upon placement of the device at the treatment site, or within 5 minutes at most from its placement. As nitric oxide is released in the gas phase, it may be achieved that no or only few residues of the NO donor are deposited in the tissue.

In preferred embodiments of the present invention, NONO'ates are applied as nitric oxide donors. NONO'ates break down into the parent amine and NO gas in an acid catalyzed manner, according to the below figure, cf. U.S. Pat. No. 6,147,068, Larry K. Keefer: Methods Enzymol, (1996) 268, 281-293, and Naunyn-Schmeideberg's Arch Pharmacol (1998) 358, 113-122.

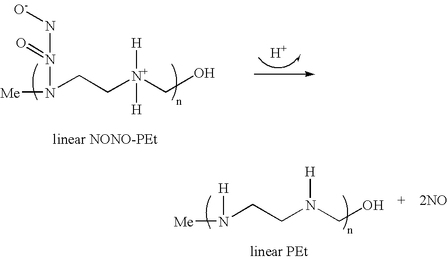

In this embodiment, NO is released within the electrospun polymer matrix. As the matrix is porous, water may enter into the matrix. The NO molecule can be transported out of the matrix and into the tissue in a number of ways and combinations hereof. In the following some scenarios are described: NO becomes dissolved in water within the matrix and transported out of the matrix by diffusion or by water flow; NO diffuse out of the matrix in gas form and becomes dissolved in water outside the matrix; NO diffuses from water into the tissue; NO diffuses all the way from the matrix in gas form into the tissue.

As illustrated in the above figure, the rate of NO liberation highly depends on the pH of the media. Thus, by addition of various amounts of an acid to the matrix, the rate of NO liberation can be controlled. As an example, the half-live of NO liberation at pH=5.0 is approximately 20 minutes whereas at pH=7.4 the half-live is approximately 10 hours. As an example, Ascorbic Acid can be used as an acidic agent for enhancing release of NO.

Various nitric oxide (NO) donor compounds and polymeric compositions capable of releasing nitric oxide have also been proposed in the prior art, e.g U.S. Pat. No. 5,691,423, U.S. Pat. No. 5,962,520, U.S. Pat. No. 5,958,427, U.S. Pat. No. 6,147,068, and U.S. Pat. No. 6,737,447 B1 (corresponding to EP 1220694 B1), all of which are incorporated herein by reference.

In preferred embodiments, the nanofibers are made from polymers which have nitric oxide donors (e.g. a diazeniumdiolate moiety) covalently bound thereto.

Polyimines represent a diverse group of polymer which may have diazeniumdiolate moieties covalently bound thereto. Polyimines include poly(alkylenimines) such as poly(ethylenimines). For example, the polymer may be a linear poly(ethylenimine) diazeniumdiolate (NONO-PEI) as disclosed in U.S. Pat. No. 6,737,447 which is hereby incorporated by reference. The loading of the nitric oxide donor onto the linear poly(ethylenimine) (PEI) can be varied so that 5-80%, e.g. 10-50%, such as 33%, of the amine groups of the PEI carry a diazeniumdiolate moiety. Depending on the applied conditions, the linear NONO-PEI can liberate various fractions of the total amount of releasable nitric oxide.

Polyamines with diazeniumdiolate moieties (in particular poly(ethylenimine) diazeniumdiolate) may advantageously be used as a polymer for the electrospinning process because such polymers typically have a suitable hydrophilicity and because the load of diazeniumdiolate moieties (and thereby the load of latent NO molecules) can be varied over a broad range, cf. the above example for NONO-PEI.

In another embodiment, the pharmaceutically active substance(s) is/are present within the polymer matrix as discrete molecules.

Within this embodiment, it the pharmaceutically active substance(s) may be contained in microparticles, such as microspheres and microcapsules. Such microparticles are in particular useful in the treatment of cancer. The microparticles may be biodegradable and may be made from a biodegradable polymer such as a polysaccharide, a polyamino acid, a poly(phosphorester) biodegradable polymer, a polymers or copolymers of glycolic acid and lactic acid, a poly(dioxanone), a poly(trimethylene carbonate)copolymer, or a poly(α-caprolactone) homopolymer or copolymer.

Alternatively, the microparticles may be non-biodegradable, such as amorphous silica, carbon, a ceramic material, a metal, or a non-biodegradable polymer.

The microparticles may be in the form of microspheres that encapsulate the pharmaceutically active substance, such as the chemotherapeutic agent. The release of the pharmaceutically active substance preferably commences after the administration.

The encapsulating microspheres may be rendered leaky for the pharmaceutically active substance by means of an electromagnetic or ultrasound shock wave.

In order to facilitate passage of a guide wire or shaft according to the invention to the treatment site along an often tortuous path, a hydrophilic layer is preferably applied to the outer surface layer. The hydrophilic layer may be provided as a separate layer of material. Alternatively, the outer surface layer may itself exhibit hydrophilic properties.

The outer surface layer may advantageously include an acidic agent, such as lactic acid or vitamin C, which acts as a catalyst for releasing the pharmaceutically active substance, e.g. nitric oxide. The acidic agent is capable of changing the ph-value at the treatment site, the release rate of nitric oxide at the treatment site varying as a function of the local ph-value. Thus, the presence of vitamin C may boost the nitric oxide release, i.e. provide a shock-like release of nitric oxide.

In general, the release of nitric oxide is described in Prevention of intimal hyperplasia after angloplasty and/or stent insertion. Or, How to mend a broken heart by Jan Harnek M D, Heart Radiology, University of Lund, Sweden, 2003.

The pharmaceutically active substance may be provided in the form of biodegradable beadings distributed between the nanofibers, the beadings being capable of releasing the pharmaceutically active substance and, in the case of biodegradable beadings, to degrade following release. Such beadings, which are described in more detail in International patent application No. PCT/DK2004/000560 which is hereby incorporated by reference in its entirety, may penetrate into the tissue at the treatment site and release the pharmaceutically active substance there. Alternatively, they may be of a size which is so small that they may be transported away, e.g. with the flow of blood, away from the treatment site.

In one embodiment of the method of producing the device, nitric oxide may be applied to the outer surface layer by exposing the outer surface layer to nitric oxide in a chamber containing pressurized nitric oxide at a pressure of, e.g. 1-5 bar, or 1.5-5 bar, or 2-5 bar.

The step of electrospinning nanofibers usually comprises feeding a fiber-forming material through-a-dispensing electrode arranged at a distance from a supporting element, whereby a plurality of strands of the fiber-forming material emerge out of said dispensing electrode. In one embodiment of the present method, the properties of the outer surface layer are controlled by controlling the fluidity of the strands when they reach the supporting element, for example by controlling the distance between the dispensing electrode and the supporting element. By controlling the fluidity of the jet, the crossing fibers can be made into a multiply connected network which is unlikely to unwind if the network broke at only one point. Also, the fluidity may enable the more fluid fibers to conform closely to the shape of the device or any other supporting element used in the electrospinning process everywhere the fibers contact the device or supporting element.

In a broadest aspect, the invention provides a medical device for insertion into the vascular system of a living being, at least a portion of the medical device being formed by electrospun nanofibers, and a method of a medical device, such as a medical tubing, such as a vascular implant, a vascular graft, stent, stent graft, embolization device or catheter for insertion into the vascular system of a living being, the method comprising the step of forming at least a portion of the medical device by electrospinning of nanofibers, which consolidate to form the medical device, or at least said portion thereof. The electrospun part of the device may e.g. be an outer surface layer which may comprise any feature of the outer surface layer of the device according to the first aspect of the invention disclosed herein.

### BRIEF DESCRIPTION OF THE DRAWINGS

Embodiments of the invention will now be further described with reference to the drawing, in which:

FIGS. 1-6 are step-by-step illustrations of an embodiment of a method for producing a medical device;

FIG. 7 shows a longitudinal side view of a stent partially coated with nanospun fibers;

FIGS. 8-10 illustrate two embodiments of embolization devices in the form of coils;

FIGS. 11 and 12 illustrate an embolization device in the form of a three-dimensional sphere;

FIG. 13 illustrates an embolization device in the form of particle, onto which there is applied filaments by electrospinning.

### DETAILED DESCRIPTION OF THE DRAWING

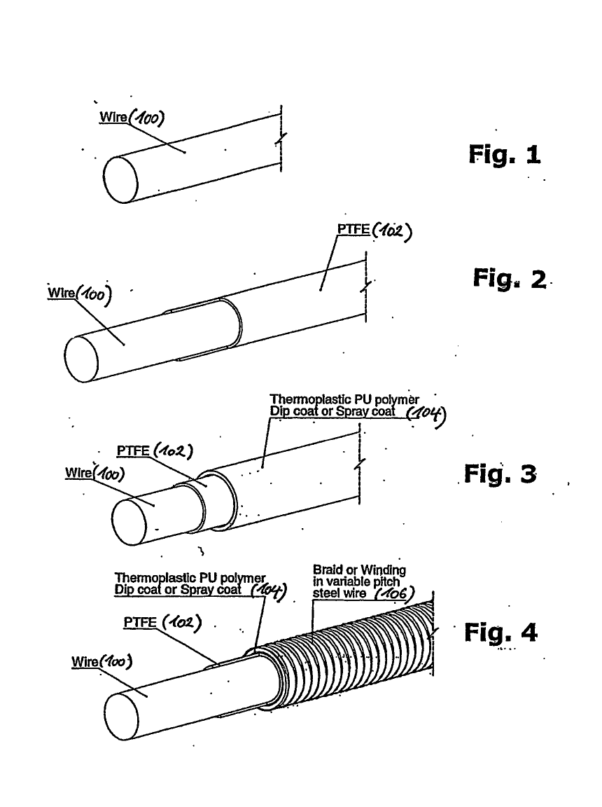

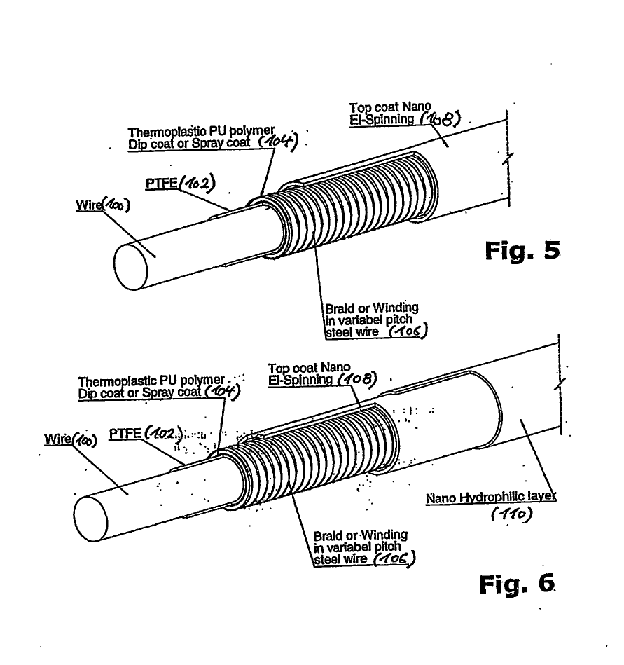

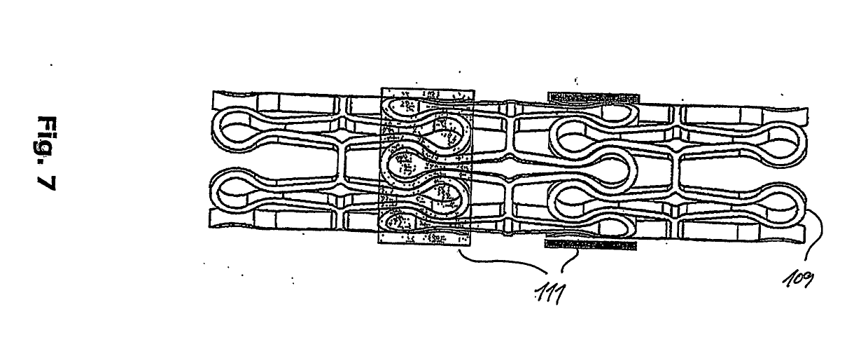

Though the invention will now be further described with reference to the tubing illustrated in FIGS. 1-6, the stent in FIG. 7 and the embolization devices of FIGS. 8-10, it will be appreciated that the below description is not limited to medical tubing, stents and embolization devices. Accordingly, any other medical device forte introduction into the vascular system of a living being may be produced as described below.

In the embodiment of FIGS. 1-6, the nanofibers are spun onto an outer surface of a core member. The core member comprises a core wire (or mandrel) 100, a layer 102 of PTFE applied to an outer surface of the core wire, a coating 104 of a thermoplastic material applied to an outer surface of the PTFE layer 102, and at least one reinforcing wire 106 applied to an outer surface of the thermoplastic coating, with the filaments of electrospun nanofibers being provided as an outer layer 108, i.e. enclosing the reinforcing wire and the thermoplastic coating. A hydrophilic layer 110 is optionally applied to an outer surface of the device, cf. FIG. 6.

Preferably, the diameter of the guide wire is at least 0.1 mm, such as in the range of 0.1 to 1.0 mm, or larger. The thermoplastic coating, which is preferably a coating of polyurethane (PU), preferably has a thickness of 5 μm to about 0.05 mm, preferably 0.01 mm±20%. The reinforcing wire(s) preferably has/have a diameter of 5 μm to about 0.05 mm, preferably 0.01 mm±20%

There may be provided one single core wire or a plurality of core wires which may be arranged side-by-side and extend in parallel. In the case of a plurality of core wires, the tubing so produced is a so-called multiple lumen tubing, with the core member being constituted by the plurality of core wires, around which the nanofibers are spun, so that the nanofibers and optionally the PTFE layer, thermoplastic layer and reinforcing wire(s) enclose the plurality of core wires. A multiple lumen tubing is for example useful in connection with pressure measurements, for example for measuring a pressure drop across stenosis. One or more passages of a multiple lumen tubing may be used for transmitting light, for example light which may be emitted through blood, thereby facilitating diagnostic procedures.

As described above, a layer of PTFE 102 may be applied to an outer surface of the core member 100. At least a portion of the surface of the layer of PTFE, such as the portion onto which the nanofibers and/or the thermoplastic coating are to be applied, may be modified for improved bonding of material to the outer surface of the PTFE layer. Preferably, such modifying comprises etching, which may for example result in a primed PTFE surface for covalent bonding or gluing. Etching may be achieved by applying a flux acid or hydroflouric acid to a surface of the PTFE layer. The layer of PTFE may be provided as a hose which is slipped over and co-extends with the core wire, or, in the case of a multiple lumen tubing, the plurality of core wires.

A coating of a thermoplastic material 104, such as polyurethan (PU), may be provided to an outer surface of the core member 100, i.e. to an outer surface of the PTFE layer 102 in case such a layer has been provided. Following the step of providing the layer of PTFE 102 and/or the step of providing the thermoplastic coating 104, one or more reinforcing wires 106 may be applied to an outer surface of the core member 100, i.e., in a preferred embodiment, to an outer surface of the polyurethane coating 104. The reinforcing wire(s) may consist of one or wires made from steel or/and wires made from yam, such as carbon filament, which may be applied by winding. Alternatively, the reinforcing wire may be applied by spinning of nanofibers, preferably by electrospinning as described above. The electrospun reinforcing wire may be formed from carbon or polymer, including polymer solutions and polymer melts. Applicable polymers are: nylon, fluoropolymers, polyolefins, polyimides, and polyesters.

While forming the medical device, or at least while forming that portion of the medical device which is formed by electrospinning, the core member 100 is preferably rotated, so as to evenly distribute the nanofibers around the outer surface of the core member.

In a preferred embodiment of the invention, nanofibers 108 are applied to the outer surface of the core member at this stage, that is preferably to the outer surface of the thermoplastic coating 104 which is optionally reinforced by the reinforcing wire(s). The electrospinning process is discussed in detail above.

A solvent, such as tetrahydroforane (THF) or isopropanol alcohol (IPA), may subsequently be applied to an outer surface of the core member, the outer surface being defined by the electrospun portion (or layer) 108 of the device. The thermoplastic coating 104 thereby at least partially dissolves in the solvent, so as to bond the reinforcing wire(s) 106 thereto. The reinforcing wire(s) 106 thereby become(s) embedded in the thermoplastic coating 104. It has been found that the step of providing the solvent results in a highly dense surface with a low surface friction, which is believed to be due to crumpling or shrinking of stretched molecules of electrospun nanofibers once the solvent is applied.

A stent graft may be produced by omitting the step of applying the solvent.

The core wire 100 (or mandrel) is removed from the device following the step of applying the solvent or prior to the step of applying solvent but subsequent to the step of applying the filament of electrospun nanofibers 108.

FIG. 7 illustrates a zig-zag corrugated stent 109 with portions of electrospun nanofilaments 111 applied to a surface thereof.

The embolization device of FIG. 8 comprises a wire which is wound into the form of a coil and coated with electrospun nanofibers. The device of FIG. 9 is a coil which is formed by a wound coil as illustrated by the cross section of FIG. 10.

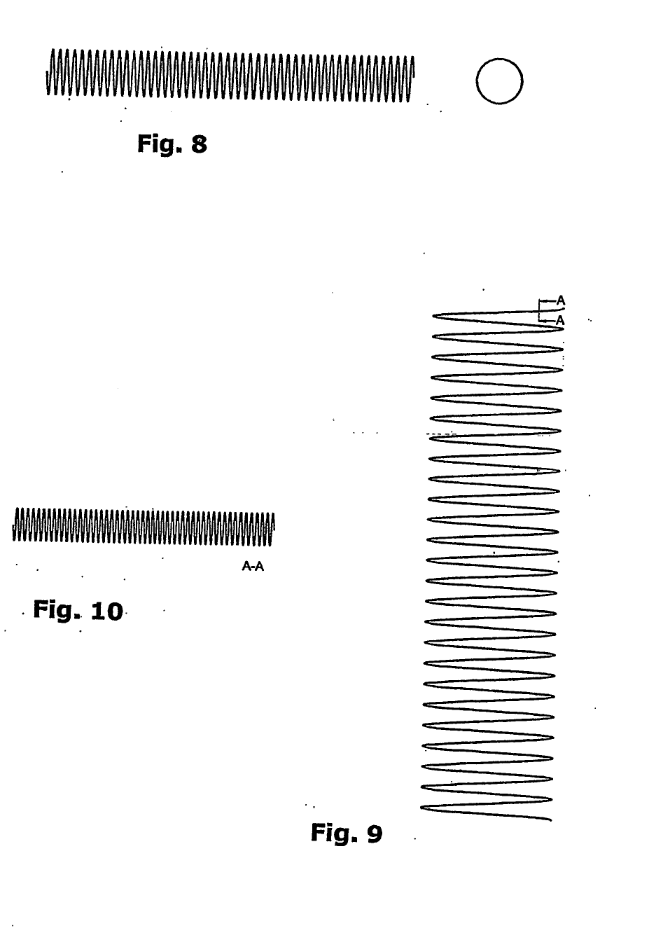

FIG. 12 illustrates an embolization device in the form of a three-dimensional sphere, produced by a method according to the invention. Electrospun nanofilaments are applied to a base element 112 which, as shown in the cross-section of FIG. 11, consists essentially of a string or coil element.

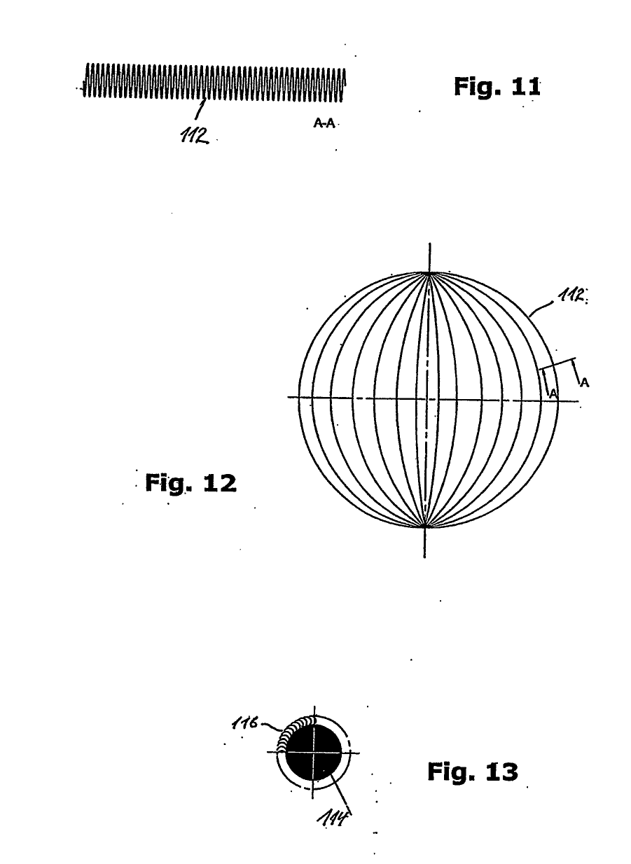

FIG. 13 illustrates an embolization device in the form of a tantalum particle 114, onto which there is applied electrospun filaments 116.

1. A medical device comprising a solid and/or non-expandable core member having an outer surface layer, which is formed by electrospun nanofibers.

2. A medical device according to claim 1, wherein the medical device is selected from the group consisting of:
a guide wire for guiding medical devices through tubular structures of a living being; and
an embolization device
a guide shaft for a micro catheter.

3. A medical device according to claim 1 or 2, wherein the outer surface layer incorporates a pharmaceutically active substance.

4. A medical device according to claim 3, wherein the pharmaceutically active substance comprises nitric oxide, and wherein the outer surface layer further comprises an acidic agent.

5. A medical device according to claim 3, wherein the pharmaceutically active substance comprises a chemotherapeutical agent.

6. A medical device according to any of claims 3-5, wherein the outer surface layer is essentially made from a polymer matrix, which contains molecules capable of releasing the at least one pharmaceutically active substance.

7. A medical device according to claim 6, wherein the outer surface layer is essentially made from a polymeric linear poly(ethylenimine) diazeniumdiolate.

8. A medical device according to any of claims 3-7, wherein the pharmaceutically active substance is provided in the form of biodegradable beadings distributed between the nanofibers.

9. A medical device according to any of claims 2-8, the medical device being an embolization device, which has an essentially spherical outer contour, and which is made from one or more coil elements, the outer surface layer being provided on the or each coil element.

10. A medical device according to any of claims 2-8, the medical device being an embolization device, wherein the core member is a particle, onto which the outer surface layer is formed.

11. A medical device according to any of claims 2-8, the medical device being an embolization device, the core member of which is made from a thrombogenic, biodegradable polymer.

12. A medical device according to claim 11, wherein the biodegradable polymer comprises collagen.

13. A medical device according to claim 11 or 12, wherein the biodegradable polymer comprises polylactid.

14. A medical device according to any of claims 11-13, wherein the biodegradable polymer comprises urethane.

15. A method of producing a medical device comprising a solid and/or non-expandable core member having an outer surface layer, the method comprising forming the outer surface layer by electrospinning of nanofibers.

16. A method according to claim 15, wherein the outer surface layer comprises a pharmaceutically active substance.

17. A method according to claim 16, wherein the pharmaceutically active substance comprises nitric oxide.

18. A method according to claim 17, wherein the outer surface layer further comprises an acidic agent.

19. A method according to claim 16, wherein the pharmaceutically active substance comprises a chemotherapeutical agent.

20. A method according to any of claims 15-19, wherein the outer surface layer is essentially made from a polymer matrix, which contains molecules capable of releasing the at least one pharmaceutically active substance.

21. A method according to claim 20, wherein the outer surface layer is essentially made from a polymeric linear poly(ethylenimine) diazeniumdiolate.

22. A method according to any of claims 15-21, wherein nitric oxide is applied to outer surface layer by exposing the outer surface layer to nitric oxide in a chamber containing pressurized nitric oxide.

23. A method according to claim 22, wherein the device is exposed to nitric oxide at a pressure of 1-5 bar in said chamber.

24. A method according to any of claims 15-23, wherein the step of electrospinning nanofibers comprises feeding a fiber-forming material through a dispensing electrode arranged at a distance from the core element, whereby a plurality of strands of the fiber-forming material emerge out of said dispensing electrode, the method comprising controlling the properties of the outer surface layer by controlling the fluidity of said strands when they reach the supporting element.

25. A method according to claim 24, wherein the fluidity of the strands when they reach the core element is controlled by controlling the distance between dispensing electrode and the core element.

26. A method according to any of claims 15-25, wherein the core member is provided on a sheet-like supporting member, and wherein the outer surface layer is formed by electrospinning of nanofibers while the core member is supported by said supporting member.

27. A method according to any of claims 15-26, wherein the outer surface layer is provided to the core member in a fluid bed.

28. A medical device for insertion into the vascular system of a living being, at least a portion of the medical device being formed by electrospun nanofibers.
